(19) United States
(12) Patent Application Publication
(10) Pub. No.: US 2006/0121609 A1
(43) Pub. Date: Jun. 8, 2006

(54) Gradient scaffolding and methods of producing the same

(76) Inventors: Ioannis V. Yannas, Newton, MA (US); Lorna J. Gibson, Jamaica Plain, MA (US); Fergal J. O'Brien, Dublin (IE); Brendan Harley, Cambridge, MA (US); Ricardo R. Brau, Cambridge, MA (US); Stephen Samouhos, Boston, MA (US); Myron Spector, Brookline, MA (US)

Correspondence Address: PEARL COHEN ZEDEK, LLP, 1500 BROADWAY 12TH FLOOR, NEW YORK, NY, 10036, US

(21) Appl. No.: 11/230,918

(22) Filed: Sep. 21, 2005

Related U.S. Application Data
(60) Provisional application No. 60/611,266, filed on Sep. 21, 2004.

(51) Int. Cl. C12N 5/08; A61F 2/02

(52) U.S. Cl. 435/395; 623/23.74

(57) ABSTRACT

This invention relates to gradient scaffolds, methods of producing the same, and methods of use thereof, in particular for applications in tissue engineering, repair and regeneration. The gradient scaffolding includes, inter-alia, scaffolds, which are varied in terms of their pore diameter, chemical composition, crosslink density, or combinations thereof, throughout the scaffolding.

### CROSS REFERENCE TO RELATED APPLICATIONS

This Application claims the benefit of U.S. Provisional Application Ser. No. 60/611,266, filed Sep. 21, 2004, which is hereby incorporated in its entirety.

### FIELD OF THE INVENTION

This invention relates to gradient scaffolding and methods of producing the same. The gradient scaffolding includes, inter-alia, scaffolds, which display controlled variation along a desired direction of one or several properties, including pore diameter, chemical composition, crosslink density, or combinations thereof.

### BACKGROUND OF THE INVENTION

One of the limitations to date in successful tissue engineering is a lack of an appropriate material and architecture whereby complex tissues may be assembled, in particular providing the ability of appropriate cells to align themselves along desired directions to form functioning tissue. Current methodology also is lacking in terms of providing an appropriate substrate that facilitates formation of tissue for regions of tissue attached to each other, where each region differs in terms of its resident cell type and composition.

Many tissues and organs are anatomically separated from neighboring tissues/organs, often by means of non-specific tissue such as fascia. Other tissues/organs, however, merge into neighboring organs and such an extension shows a progressive change in structure, i.e., it forms a gradient in one or more properties, conferring thereby important new functional properties to the tissue. Attachment of the two tissues/organs by such “connector” tissues in the form of gradient structures generates a new physiological function that is lost when the connection between the two tissues/organs is severed, e.g., following trauma Examples of such tissue include tendon, ligament and articular cartilage, associated with the musculoskeletal system. In each of these examples, mechanical forces essential to the healthy functioning of the body are transmitted from one organ to the ached “connector” tissue, and in turn, to an organ attached thereto.

When two differentiated tissues or organs are attached by a third connector tissue, the connector typically comprises three types of tissue. At each end, the connector is typically structurally or functionally identical to the tissues or organs with which each end will connect. The intermediate part of the connector typically has a distinct and unique structure or architecture, which is related to its mechanical function, including the mechanical coupling of the two tissues with which it is connected

The musculoskeletal connective tissues can frequently be injured traumatically. In addition to healing the tissue itself, via stimulation of its reparative (scar formation) or regenerative function, for successful functioning of the tissue, and in order to recover of the entire organ it is necessary to heal appropriately not only the end organs but the connector tissue as well. For example, when tendon and ligament are injured, these structures as well as bone to which they are attached must heal; however, to regain function of the injured limb it is necessary for the tissue that keeps them attached to bone to heal appropriately as well. Scaffolding which induces the repair must also therefore stimulate synthesis of new connector tissue, which extends from the reference tissue/organ to the neighboring tissue/organ with which it will be attached. Because the connector tissue is typically comprised of at least three different kinds of tissue, spatially arranged in order to maintain the appropriate connections, then the scaffold must stimulate synthesis of the three tissues, and the synthesis must provide for the appropriate architecture of the connector.

While scaffolding exists in the art, the material used to date induces regeneration of a single tissue type. The regenerative activity of the scaffolds depends quite sensitively on the average pore diameter, chemical composition and cross-link density, and current art emphasizes uniformity of one of these properties throughout the scaffolding material. A scaffold that induces regeneration of a tissue has an architecture that is intimately related, being almost a replica of, the architecture of the stroma (connective tissue) in the tissue undergoing regeneration. A scaffold that is characterized by uniform structure throughout, as is currently practiced, will not readily accommodate the synthesis of connector tissue/organs, which necessarily comprise different tissue types, and therefore require non-uniform makeup for successful tissue regeneration.

### SUMMARY OF THE INVENTION

In one embodiment, the invention provides a solid, biocompatible gradient scaffold, which in another embodiment is porous

According to this aspect of the invention, and in one embodiment, the solid polymer comprises at least one synthetic or natural polymer, ceramic, metal, extracellular matrix protein or an analogue thereof. In another embodiment, the scaffold is non-uniformly porous, or in another embodiment, the pores within the scaffold are of a non-uniform average diameter. In another embodiment, the average diameter of said pores varies as a function of its spatial organization in said scaffold, or in another embodiment, average diameter of said pores varies as a function of the pore size distribution along an arbitrary axis of said scaffold. In another embodiment, the scaffold varies in its average pore diameter or distribution thereof, concentration of components, cross-link density, or a combination thereof. In another embodiment, the average diameter of said pores ranges from 0 001-500 μm.

In another embodiment, this invention provides a process for preparing a non-uniformly porous, solid, biocompatible gradient scaffold, comprising at least one extracellular matrix component or an analog thereof, comprising the steps of:
(a) Freeze-drying a solution of at least one extracellular matrix component or an analog thereof, under conditions producing a gradient in the freezing temperature; and
(b) Sublimating ice-crystals formed within the slurry in step (a), prior to achievement of thermal equilibrium during said freeze-drying;
   Wherein ice-crystals are formed along a gradient as a function of the gradient freezing temperature, whereby sublimation of said ice-crystals results in the formation of pores arranged along said gradient.

According to this aspect of the invention, and in one embodiment, the extracellular matrix component comprises a collagen, a glycosaminoglycan, or a combination thereof. In another embodiment, the process further comprises the steps of moistening at least one region within the scaffold formed in step (b) and exposing the moistened region to drying, under conditions comprising atmospheric pressure, such that exposing the moistened region to drying results in pore collapse in said region. In another embodiment, scaffold produced comprises regions devoid of pores. In another embodiment, moistening the legion is conducted such that following exposure to drying, the regions devoid of pores assume a particular geometry. In another embodiment, the regions ale impenetrable to molecules with a radius of gyration or effective diameter of at least 1000 Da in size.

In another embodiment, the process further comprises the step of exposing the scaffold to a gradient of solutions, which are increased in their salt concentration. In one embodiment, exposure to the salt results in selective solubilization of at least one extracellular matrix component in said scaffold. In another embodiment, solubilization of at least one extracellular matrix component increases as a function of increasing salt concentration.

In another embodiment, the process further comprises the step of exposing the scaffold to a gradient of solutions, which are increased in their concentration of an enzyme, which degrades or solubilizes at least one extracellular matrix component. According to this aspect of the invention, and in one embodiment, digestion of at least one extracellular matrix component increases as a function of increasing enzyme concentration. In one embodiment, the enzyme is a collagenase, a glycosidase, or a combination thereof. In another embodiment, the enzyme concentration is at a range between 0.001-500 U/ml.

In another embodiment, the process further comprises the step of exposing the scaffold to a temperature gradient. According to this aspect of the invention, and in one embodiment, the temperature gradient is a range between 25-200° C. In another embodiment, exposing the scaffold to a temperature gradient, results in the creation of a gradient in crosslink density in said scaffold.

In another embodiment, the process further comprises the step of exposing the scaffold to a gradient of solutions, which are increased in their concentration of cross-linking agent. According to this aspect of the invention, and in one embodiment, exposure to the cross-linking agent results in the creation of a gradient in crosslink density in the scaffold. In one embodiment, the cross-linking agent is glutaraldehyde, formaldehyde, paraformaldehyde, formalin, (1 ethyl 3-(3dimethyl aminopropyl)carbodiimide (EDAC), or UV light, or a combination thereof.

In another embodiment, this invention provides a process for preparing a non-uniformly porous, solid, biocompatible scaffold, comprising at least one extracellular matrix component or an analog thereof, comprising the steps of:
(a) Freeze-drying a solution of two or more extracellular matrix components or analogs thereof;
(b) Sublimating ice-crystals formed within the slurry in step (a) to produce a scaffold with uniformly distributed pores;
(c) Moistening at least one legion within said scaffold formed in step (b); and
(d) Exposing the moistened region produced in step (c) to drying, under conditions of atmospheric pressure
   Wherein exposing said moistened region to drying results in pore collapse in said region, thereby producing a non-uniformly porous, solid, biocompatible scaffold

According to this aspect of the invention, and in one embodiment, the process further comprises the step of exposing the scaffold to a gradient of solutions, which are increased in their salt concentration. In another embodiment, the process further comprises the step of exposing the scaffold to a gradient of solutions, which are increased in their concentration of an enzyme, which degrades or solubilizes at least one extracellular matrix component. In another embodiment, the process further comprises the step of exposing the scaffold to a temperature gradient resulting in the creation of a gradient in crosslink density in the scaffold. In another embodiment, the process further comprises the step of exposing the scaffold to a gradient of solutions, which are increased in their concentration of cross-linking agent.

In another embodiment, this invention provides a process for preparing a solid, biocompatible gradient scaffold, comprising at least one extracellular matrix component or an analog thereof, comprising the steps of:
(a) Preparing a solution of a graft copolymer of two or more extracellular matrix components or analogs thereof;
(b) Freeze-drying the solution in step (a) to yield a porous, solid scaffold of uniform composition; and
(c) Exposing the scaffold formed in step (b) to a gradient of solutions, which are increased in their salt concentration;
   Wherein exposing said scaffold to said gradient of solutions, which are increased in their salt concentration results in selective solubilization of at least one extracellular matrix component, and said solubilization increases as a function of increased sulfate salt concentration, thereby producing a solid, biocompatible gradient scaffold.

According to this aspect of the invention, and in one embodiment, the process further comprises the step of exposing the scaffold to a gradient of solutions, which are increased in their concentration of an enzyme, which degrades or solubilizes at least one extracellular matrix component. In another embodiment, the process further comprises the step of exposing the scaffold to a temperature gradient. In another embodiment, the process further comprises the step of exposing the scaffold to a gradient of solutions, which ale increased in their concentration of cross-linking agent.

In another embodiment, this invention provides a process fox preparing a porous, solid, biocompatible gradient scaffold, comprising one or more extracellular matrix components or analogs thereof, comprising the steps of:
(a) Preparing a solution of a graft copolymer of one or more extracellular matrix components or analogs thereof;
(b) Freeze-drying the solution in step (a) to yield a porous, solid scaffold of uniform composition; and
(c) Exposing the scaffold formed in step (b) to a gradient of solutions, which ale increased in their concentration of an enzyme which digests at least one of said two or more extracellular matrix components
   Wherein exposing said scaffold to said gradient of solutions, results in selective digestion of at least one of said two or more extracellular matrix components, and said digestion increases as a function of increasing enzyme concentration, thereby producing a porous, solid biocompatible gradient scaffold.

According to this aspect of the invention, and in one embodiment, the process further comprises the step of exposing the scaffold to a temperature gradient. In another embodiment, the process further comprises the step of exposing the scaffold to a gradient of solutions, which are increased in their concentration of cross-linking agent.

In another embodiment, this invention provides a process for preparing a solid, porous, biocompatible gradient scaffold, comprising one or more extracellular matrix components or analogs thereof, comprising the steps of:
(a) Preparing a solution of a graft copolymer of one or more extracellular matrix components or analogs thereof;
(b) Freeze-drying the solution in step (a) to yield a solid scaffold of uniform composition; and
(c) Exposing the scaffold formed in step (b) to a temperature gradient
   Wherein exposing said scaffold to said temperature gradient, results in the creation of a gradient in crosslink density in said scaffold, thereby producing a solid, porous biocompatible gradient scaffold.

According to this aspect of the invention, and in one embodiment, the process further comprises exposing the scaffold to a gradient of solutions, which are increased in their concentration of cross-linking agent

In another embodiment, this invention provides a process for preparing a solid, porous biocompatible gradient scaffold, comprising at least one extracellular matrix component or analogs thereof, comprising the steps of:
(a) Preparing a solution of a graft copolymer of one or more extracellular matrix components or analogs thereof;
(b) Freeze-dying the solution in step (a) to yield a solid, porous scaffold of uniform composition; and
(c) Exposing the scaffold formed in step (b) to a gradient of solutions, which are increased in their concentration of cross-linking agent
   Wherein exposing said scaffold to said gradient of solutions, which are increased in their concentration of cross-linking agent, results in the creation of a gradient in crosslink density in said scaffold, thereby producing a solid, porous biocompatible gradient scaffold

In another embodiment, this invention provides a solid, porous biocompatible gradient scaffold, prepared according to a process of this invention.

In another embodiment, this invention provides a method of organ or tissue engineering in a subject, comprising the step of implanting a scaffold of this invention in a subject.

In another embodiment, this invention provides a method of organ or tissue repair or regeneration in a subject, comprising the step of implanting a scaffold of this invention in a subject

According to these aspects of the invention, and in one embodiment, the method further comprises the step of implanting cells in the subject. In one embodiment, the cells are seeded on said scaffold. In another embodiment, the cells are stem or progenitor cells. In another embodiment, the method further comprises the step of administering cytokines, growth factors, hormones or a combination thereof to the subject. In another embodiment, the engineered organ or tissue is comprised of heterogeneous cell types. In another embodiment, the engineered organ or tissue is a connector organ or tissue, which in another embodiment, is a tendon or ligament.

### DETAILED EMBODIMENTS OF THE INVENTION

The invention is directed to solid gradient scaffolds, methods of producing the same, and therapeutic applications arising from their utilization.

Tissue engineering, repair and regeneration has been significantly hampered due to a lack of appropriate material and architecture whereby complex tissue may be assembled, in particular providing the ability of appropriate cells, including multiple cell types, to align themselves in three dimensions to form functioning tissue. Current methodology is also lacking in terms of providing an appropriate substrate that facilitates formation of tissue for regions of tissue attached to each other, where each region differs in terms of its resident cell type and composition.

In one embodiment, the invention provides solid, porous biocompatible gradient scaffold, comprising a polymer.

The term “scaffold”, in one embodiment, refers to a three dimensional structure, that serves as a support fox and/or incorporates cells, biomolecules, or combinations thereof. In one embodiment, a scaffold provides a support for the repair, regeneration or generation of a tissue or organ

The term “gradient scaffold”, in one embodiment, refers to a scaffold that is comprised of a material which varies in terms of, in one embodiment, the concentration of components of which the scaffold is comprised, or in another embodiment, its porosity (which may be reflected in other embodiments in terms of, pore size, pore shape, percent porosity), or in another embodiment, its cross-link density, or in another embodiment, its density, throughout the scaffold. In another embodiment, the term “gradient scaffold” refers to scaffold comprised of material with varying pore diameter throughout the scaffold.

In one embodiment, the gradient scaffold is characterized by a progressively changing pore volume fraction, ranging from a pore fraction of 0 to 0.999.

In one embodiment, the mean pore diameter may range between 0.001-500 μm. In one embodiment, the mean pore diameter may range between 0.001-0.01 μm, or in another embodiment, between 0.001-500 μm, or in another embodiment, between 0.001-0.1 μm, or in another embodiment, between 0.1-1 μm, or in another embodiment, between 0.001-500 μm, or in another embodiment, between 0.1-10 μm, or in another embodiment, between 1-10 μm, in another embodiment, between 1-25 μm, or in another embodiment, between 10-50 μm, or in another embodiment, between 0.001-500 μm, or in another embodiment, between 10-74 μm, or in another embodiment, between 25-100 μm, or in another embodiment, between 100-250 μm, or in another embodiment, between 100-500 μm.

In one embodiment, the term “gradient scaffold” refers to a scaffold wherein the pores formed are of a non uniform average diameter. In another embodiment, the term “gradient scaffold” refers to a scaffold wherein the pores formed are of a uniform average diameter, which are distributed non-uniformly, throughout the scaffolding material.

In another embodiment, the term “gradient scaffold” refers to a varying concentration of the solid polymer comprising the scaffolding. In one embodiment, the concentration varies throughout the scaffolding. In another embodiment, the solid polymer concentration varies along at least one axis of the scaffold. In another embodiment, the solid polymer concentration is varied at specific positions in the scaffolding, which, in another embodiment, facilitates cell adhesion

In one embodiment, the term “gradient scaffold” refers to a material utilized to synthesize one or more tissues in close proximity to each other.

In one embodiment, the term “biocompatible” refers to products that break down not simply into basic elements, but into elements that are actually beneficial or not harmful to the subject or his/its environment. In another embodiment, the term “biocompatible” refers to the property of not inducing fibrosis, inflammatory response, host rejection response, or cell adhesion, following exposure of the scaffold to a subject or cell in said subject. In another embodiment, the term “biocompatible” refers to any substance or compound that has minimal (i.e., no significant difference is seen compared to a control), if any, effect on sounding cells or tissue exposed to the scaffold in a direct or indirect manner.

In one embodiment, the polymers of this invention may be copolymers. In another embodiment, the polymers of this invention may be homo- or, in another embodiment heteropolymers. In another embodiment, the polymers of this invention are synthetic, or, in another embodiment, the polymers are natural polymers. In another embodiment, the polymers of this invention are free radical random copolymers, or, in another embodiment, graft copolymers. In one embodiment, the polymers may comprise proteins, peptides or nucleic acids.

In one embodiment, the polymers of this invention may comprise hydrophobic polymers such as polycarbonate, polyester, polypropylene, polyethylene, polystyrene, polytetrafluoroethylene, polyvinyl chloride, polyamide, polyacrylate, polyurethane, polyvinyl alcohol polyurethane, polycaprolactone, polylactide, polyglycolide or copolymers of any thereof. In another embodiment, the polymers may comprise siloxanes such as 2,4,6,8-tetramethylcyclotetrasiloxane; natural and/or artificial rubbers; glass; metals including stainless steel or graphite, or combinations thereof.

In one embodiment, the polymers of this invention may comprise hydrophilic polymers such as a hydrophilic diol, a hydrophilic diamine or a combination thereof. The hydrophilic diol can be a poly(alkylene)glycol, a polyester-based polyol, or a polycarbonate polyol. In one embodiment, the term “poly(alkylene)glycol” refers to polymers of lower alkylene glycols such as poly(ethylene)glycol, poly(propylene)glycol and polytetramethylene ether glycol (PIMEG). The term “polyester-based polyol” refers to a polymer in which the R group is a lower alkylene group such as ethylene, 1,3-propylene, 1,2-propylene, 1,4-butylene, 2,2-dimethyl-1,3-propylene, and the like. One of skill in the art will also understand that the diester portion of the polymer can also vary. For example, the present invention also contemplates the use of succinic acid esters, glutaric acid esters and the like. The term “polycarbonate polyol” refers those polymers having hydroxyl functionality at the chain termini and ether and carbonate functionality within the polymer chain. The alkyl portion of the polymer may, in other embodiments, be composed of C2 to C4 aliphatic radicals, or in some embodiments, longer chain aliphatic radicals, cycloaliphatic radicals or aromatic radicals. In one embodiment, the term “hydrophilic diamines” refers to any of the above hydrophilic diols in which the terminal hydroxyl groups have been replaced by reactive amine groups or in which the terminal hydroxyl groups have been derivatized to produce an extended chain having terminal amine groups. For example, in one embodiment, a hydrophilic diamine is a “diamino poly(oxyalkylene)” which is poly(alkylene)glycol in which the terminal hydroxyl groups are replaced with amino groups. The term “diamino poly(oxyalkylene)” also refers to poly(alkylene)glycols which have aminoalkyl ether groups at the chain termini. One example of a suitable diamino poly(oxyalkylene) is poly(propylene glycol) bis(2-aminopropyl ether). A number of diamino poly(oxyalkylenes) are available having different average molecular weights and are sold as Jeffamines™ (for example, Jeffamines 230, Jeffamine 600, Jeffamine 900 and Jeffamine 2000). These polymers can be obtained, for example, from Aldrich Chemical Company. Literature methods can be employed for their synthesis, as well.

In another embodiment, the polymers of this invention may comprise Prolene™, nylon, polypropylene, Deklene™, polyester or any combination thereof.

In another embodiment, the polymers of this invention may comprise silicone polymers. In one embodiment, the silicone polymers may be linear. In one embodiment, the silicone polymer is a polydimethylsiloxane having two reactive functional groups (i.e., a functionality of 2). The functional groups can be, for example, hydroxyl groups, amino groups or carboxylic acid groups. In some embodiments, combinations of silicone polymers can be used in which a first portion comprises hydroxyl groups and a second portion comprises amino groups. In one embodiment, the functional groups are positioned at the chain termini of the silicone polymer. A number of suitable silicone polymers are commercially available from such sources as Dow Chemical Company (Midland, Mich., USA) and General Electric Company (Silicones Division, Schenectady, N.Y., USA). Still others can be prepared by general synthetic methods, beginning with commercially available siloxanes (United Chemical Technologies, Bristol Pa., USA). The silicone polymers, in other embodiments, may have a molecular weight of from about 400 to about 10,000, or in another embodiment, from about 2000 to about 4000.

In another, embodiment, the polymers of this invention may comprise extracellular matrix components, such as hyaluronic acid and/or its salts, such as sodium hyaluronate; glycosaminoglycans such as dermatan sulfate, heparan sulfate, chondroiton sulfate and/or keratan sulfate; mucinous glycoproteins (e.g., lubricin), vitronectin, tribonectins, surface-active phospholipids, rooster comb hyaluronate. In some embodiments, the extracellular matrix components may be obtained from commercial sources, such as ARIHREASE™ high molecular weight sodium hyaluronate; SYNVISC® Hylan G-F 20; HYLAGAN® sodium hyaluronate; HEALON® sodium hyaluronate and SIGMA® chondroitin 6-sulfate.

In another embodiment, the polymers may comprise biopolymers such as, for example, collagen. In another embodiment, the polymers may comprise biocompatible polymers such as polyesters of [alpha]-hydroxycarboxylic acids, such as poly(L-lactide) (PLLA) and polyglycolide (PGA); poly-p-dioxanone (PDO); polycaprolactone (PCL); polyvinyl alcohol (PVA); polyethylene oxide (PEO); polymers disclosed in U.S. Pat. Nos. 6,333,029 and 6,355,699; and any other bioresorbable and biocompatible polymer, co-polymer or mixture of polymers or co-polymers described herein.

In one embodiment, the polymer will comprise a polyurea, a polyurethane or a polyurethane polyurea combination. In one embodiment, such polymers may be formed by combining diisocyanates with alcohols and/or amines. For example, combining isophorone diisocyanate with PEG 600 and 1,4-diaminobutane under polymerizing conditions provides a polyurethane/polyurea composition having both urethane (carbamate) linkages and urea linkages.

In another embodiment, the polymers comprising extracellular matrix components may be purified from tissue, by means well known in the art. For example, if collagen is desired, in one embodiment, the naturally occurring extracellular matrix can be treated to remove substantially all materials other than collagen. The purification may be carried out to substantially remove glycoproteins, glycosaminoglycans, proteoglycans, lipids, non-collagenous proteins and nucleic acid (DNA or RNA), by known methods

In another embodiment, the polymer may comprise Type I collagen, Type II collagen, Type IV collagen, gelatin, agarose, cell-contracted collagen containing proteoglycans, glycosaminoglycans or glycoproteins, fibronectin, laminin, elastin, fibrin, synthetic polymeric fibers made of poly-acids such as polylactic, polyglycolic or polyamino acids, polycaprolactones, polyamino acids, polypeptide gel, copolymers thereof and/or combinations thereof. In one embodiment, the scaffold will be made of such materials so as to be biodegradable.

In another embodiment, the solid polymers of this invention may be inorganic, yet be biocompatible, such as, for example, hydroxyapatite, all calcium phosphates, alpha-tricalcium phosphate, beta-tricalcium phosphate, calcium carbonate, barium carbonate, calcium sulfate, barium sulfate, polymorphs of calcium phosphate, ceramic particles, or combinations thereof.

In another embodiment, the polymers may comprise a functional group, which enables linkage formation with other molecules of interest, some examples of which are provided further hereinbelow. In one embodiment, the functional group is one, which is suitable for hydrogen bonding (e.g., hydroxyl groups, amino groups, ether linkages, carboxylic acids and esters, and the like).

In another embodiment, functional groups may comprise an organic acid group. In one embodiment, the term “organic acid group” is meant to include any groupings which contain an organic acidic ionizable hydrogen, such as carboxylic and sulfonic acid groups. The expression “organic acid functional groups” is meant to include any groups which function in a similar manner to organic acid groups under the reaction conditions, for instance metal salts of such acid groups, particularly alkali metal salts like lithium, sodium and potassium salts, and alkaline earth metal salts like calcium or magnesium salts, and quaternary amine salts of such acid groups, particularly quaternary ammonium salts.

In another embodiment, functional groups may comprise acid hydrolyzable bonds including ortho-ester and amide groups. In another embodiment, functional groups may comprise base-hydrolyzable bonds including alpha-ester and anhydride groups. In another embodiment, functional groups may comprise both acid- and base-hydrolyzable bonds including carbonate, ester, and iminocarbonate groups. In another embodiment, fractional groups may comprise labile bonds, which are known in the art and can be readily employed in the methods/processes and scaffolds described herein (see, e.g. Peterson et al., Biochem. Biophys. Res. Comm, 200(3): 1586-159 (1994) 1 and Freel et al., J. Med. Chem. 43: 4319-4327 (2000)).

In another embodiment, the scaffold further comprises a pH-modifying compound. In one embodiment, the term “pH-modifying” refers to an ability of the compound to change the pH of an aqueous environment when the compound is placed in or dissolved in that environment. The pH-modifying compound, in another embodiment, is capable of accelerating the hydrolysis of the hydrolyzable bonds in the polymer upon exposure of the polymer to moisture and/or, heat. In one embodiment, the pH-modifying compound is substantially water-insoluble. Suitable substantially water-insoluble pH-modifying compounds may include substantially water-insoluble acids and bases. Inorganic and organic acids or bases may be used, in other embodiments.

In another embodiment, the scaffold is non-uniformly porous. In one embodiment, the term “porous” refers to a substrate that comprises holes or voids, rendering the material permeable. In one embodiment, non-uniformly porous scaffolds allow for permeability at some regions, and not others, within the scaffold, or in another embodiment, the extent of permeability differs within the scaffold

In one embodiment, the pores within the scaffold are of a non-uniform average diameter. In another embodiment, the average diameter of said pores varies as a function of its spatial organization in said scaffold, or in another embodiment, average diameter of said poxes varies as a function of the pore size distribution along an arbitrary axis of said scaffold.

In one embodiment, scaffolds that are non-uniformly porous are especially suited for tissue engineering, repair or regeneration, wherein the tissue is a connector tissue, or wherein the scaffold is utilized to engineer, repair or regenerate two or three, or more, tissues in close proximity to one another. A difference in porosity may facilitate migration of different cell types to the appropriate regions of the scaffold, in one embodiment. In another embodiment, a difference in porosity may facilitate development of appropriate cell-to-cell connections among the cell types comprising the scaffold, required for appropriate structuring of the developing/repairing/regenerating tissue. For example, dendrites or cell processes extension may be accommodated more appropriately via the varied porosity of the scaffolding material. In another embodiment, the permeability differences in the scaffolding material may prevent and enhance protein penetrance, wherein penetration is a function of molecular size, such that the lack of uniform porosity serves as a molecular sieve. It is to be understood that the gradient scaffolding of this invention may be used any purpose for which non-uniform porosity is desired, and is to be considered as part of this invention.

In another embodiment, the scaffold varies in its average pore diameter and/or distribution thereof. In another embodiment, the average diameter of the pores varies as a function of its spatial organization in said scaffold. In another embodiment, the average diameter of the pores varies as a function of the pore size distribution along an arbitrary axis of the scaffold. In another embodiment, the scaffold comprises regions devoid of pores. In another embodiment, the regions are impenetrable to molecules greater than 1000 Da in size.

In another embodiment, the scaffold varies in terms of its polymer concentration, or concentration of and component of the scaffold, including biomolecules and/or cells incorporated within the scaffold.

In one embodiment, as described herein, other molecules may be incorporated within the scaffold, which may, in another embodiment, be attached via a functional group, as herein described. In another embodiment, the molecule is conjugated directly to the scaffold.

In one embodiment, one or more biomolecules may be incorporated in the scaffold. The biomolecules may comprise, in other embodiments, drugs, hormones, antibiotics, antimicrobial substances, dyes, radioactive substances, fluorescent substances, silicone elastomers, acetal, polyurethanes, radiopaque filaments or substances, anti-bacterial substances, chemicals or agents, including any combinations thereof. The substances may be used to enhance treatment effects, reduce the potential for implantable article erosion or rejection by the body, enhance visualization, indicate proper orientation, resist infection, promote healing, increase softness or any other desirable effect.

In another embodiment, the biomolecule may comprise chemotactic agents; antibiotics, steroidal or non-steroidal analgesics, anti-inflammatories, immunosuppressants, anticancer drugs, various proteins (e.g., short chain peptides, bone morphogenic proteins, glycoprotein and lipoprotein); cell attachment mediators; biologically active ligands; integrin binding sequence; ligands; various growth and/or differentiation agents (e.g., epidermal growth factor, IGF-I, IGF-II, TGF-β I-III, growth and differentiation factors, vascular endothelial growth factors, fibroblast growth factors, platelet derived growth factors, insulin derived growth factor and transforming growth factors, parathyroid hormone, parathyroid hormone related peptide, bFGF; TGFβ superfamily factors; BMP-2; BMP-4; BMP-6; BMP-12; sonic hedgehog; GDF5; GDF6; GDF8; PDGF); small molecules that affect the upregulation of specific growth factors; tenascin-C; hyaluronic acid; chondroitin sulfate; fibronectin; decorin; thromboelastin; thrombin-derived peptides; heparin-binding domains; heparin; heparan sulfate; DNA fragments, DNA plasmids, or any combination thereof.

In another embodiment, the scaffold may comprise one or more of the following: bone (auto graft, allograft, and xenograft) and/or derivates of bone; cartilage (autograft, allograft and xenograft), including, for example, meniscal tissue, and/or derivatives; ligament (autograft, allograft and xenograft) and/or derivatives; derivatives of intestinal tissue (autograft, allograft and xenograft), including for example submucosa; derivatives of stomach tissue (autograft, allograft and xenograft), including for example submucosa; derivatives of bladder tissue (autograft, allograft and xenograft), including for example submucosa; derivatives of alimentary tissue (autograft, allograft and xenograft), including for example submucosa; derivatives of respiratory tissue (autograft, allograft and xenograft), including for example submucosa; derivatives of genital tissue (autograft, allograft and xenograft), including for example submucosa; derivatives of liver tissue (autograft, allograft and xenograft), including for example liver basement membrane; derivatives of skin tissue; platelet rich plasma (PRP), platelet poor plasma, bone marrow aspirate, demineralized bone matrix, insulin derived growth factor, whole blood, fibrin or blood clot.

In another embodiment, the scaffolds may comprise cells. In one embodiment, the cells may include one or more of the following: chondrocytes; fibrochondrocytes; osteocytes; osteoblasts; osteoclasts; synoviocytes; bone marrow cells; mesenchymal cells; stromal cells; stem cells; embryonic stem cells; precursor cells derived from adipose tissue; peripheral blood progenitor cells; stem cells isolated from adult tissue; genetically transformed cells; a combination of chondrocytes and other cells; a combination of osteocytes and other cells; a combination of synoviocytes and other cells; a combination of bone marrow cells and other cells; a combination of mesenchymal cells and other cells; a combination of stromal cells and other cells; a combination of stem cells and other cells; a combination of embryonic stem cells and other cells; a combination of precursor cells isolated from adult tissue and other cells; a combination of peripheral blood progenitor cells and other cells; a combination of stem cells isolated from adult tissue and other cells; and a combination of genetically transformed cells and other cells.

In one embodiment, the scaffold varies in terms of its cross-link density. In another embodiment, cross-link density varies in the scaffold, as a function of spatial organization of the components in said scaffold.

In another embodiment, this invention provides a process for preparing a non-uniformly porous, solid, biocompatible gradient scaffold, comprising at least one extracellular matrix component or an analog thereof, comprising the steps of:
(a) Freeze-drying a solution of at least one extracellular matrix component or an analog thereof, under conditions producing a gradient in the freezing temperature; and
(b) Sublimating ice-crystals formed within the slurry in step (a), prior to achievement of thermal equilibrium during said freeze-drying;
   Wherein ice-crystals are formed along a gradient as a function of the gradient freezing temperature, whereby sublimation of said ice-crystals results in the formation of pores arranged along said gradient.

In one embodiment, scaffolds are prepared according to the processes of this invention, in a highly porous form, by freeze-drying and sublimating the material. This can be accomplished by any number of means well known to one skilled in the art, such as, for example, that disclosed in U.S. Pat. No. 4,522,753 to Dagalakis, et al. For examples, porous gradient scaffolds may be accomplished by lyophilization. In one embodiment, extracellular matrix material may be suspended in a liquid. The suspension is then frozen and subsequently lyophilized. Freezing the suspension causes the formation of ice crystals from the liquid. These ice crystals are then sublimed under vacuum during the lyophilization process thereby leaving interstices in the material in the spaces previously occupied by the ice crystals. The material density and pore size of the resultant scaffold may be varied by controlling, in other embodiments, the rate of freezing of the suspension and/or the amount of water in which the extracellular matrix material is suspended at the initiation of the freezing process.

For instance, to produce scaffolds having a relatively large, uniform pore size and a relatively low material density, the extracellular matrix suspension may be frozen at a slow, controlled rate (e.g., −1° C./min or less) to a temperature of about −20° C., followed by lyophilization of the resultant mass. To produce scaffolds having a relatively small uniform pore size and a relatively high material density, the extracellular matrix material may be tightly compacted by centrifuging the material to remove a portion of the liquid (e.g., water) in a substantially uniform manner prior to freezing. Thereafter, the resultant mass of extracellular matrix material is flash-frozen using liquid nitrogen followed by lyophilization of the mass. To produce scaffolds having a moderate uniform pore size and a moderate material density, the extracellular matrix material is frozen at a relatively fast rate (e.g., >−1° C./min) to a temperature in the range of −20 to −40° C. followed by lyophilization of the mass.

According to this aspect of the invention, and in one embodiment, in order to produce gradient scaffolding of this invention, the freezing rate is controlled, such that a thermal gradient is created within the scaffold, during its formation. For example, a slurry of interest comprising polymers as described and/or exemplified herein, may be inserted in a supercooled silicone oil bath, as described by Loree et al., (1989) Proc. 15^{th} Annual Northeast Bioeng. Conf., pp 53-54). According to this aspect, in one embodiment, the container is only partially immersed, and is not completely submerged in the bath, such that a freezing front which travels up the length of the container is created, thereby creating a temperature gradient within the slurry.

In one embodiment, the gradient is preserved by halting the freezing process prior to achieving thermal equilibrium The means for determining the time to achieving thermal equilibrium in a slurry thus immersed, when in a container with a given geometry, will be readily understood by one skilled in the art. Upon achieving the desired temperature gradient, the slurry, in one embodiment, is removed from the bath and subjected to freeze-drying. Upon sublimation, the remaining material is the scaffolding comprising the polymer, with a gradient in its average pore diameter.

In another embodiment, a gradient in freezing rate of the scaffold is generated with the use of a graded thermal insulation layer between the container, which contains the scaffold components, and a shelf in a freezer on which the container is placed. In one embodiment, a gradient in the thermal insulation layer is constructed via any number of means, well known in the art, such as, for example, the construction of a thicker region in the layer along a particular direction, or in another embodiment, by varying thermal conductivity in the layer. The latter may be accomplished via use of, for example, aluminum and copper, or plexiglass and aluminum, and others, all of which represent embodiments of the present invention.

According to this aspect of the invention, and in one embodiment, the extracellular matrix component comprises a collagen, a glycosaminoglycan, or a combination thereof. It is to be understood that any embodiment listed herein, with regard to the scaffolding, is, where applicable, to be considered as an embodiment of the processing described herein, for preparing the gradient scaffolds of this invention.

In another embodiment, the process further comprises the steps of moistening at least one region within the scaffold formed in step (b) and exposing the moistened region to drying, under appropriate conditions known to those skilled in the art such as atmospheric pressure, such that exposing the moistened region to drying results in pore collapse in said region. In another embodiment, scaffold produced comprises regions devoid of pores. In another embodiment, moistening the region is conducted such that following exposure to drying, the regions devoid of pores assume a particular geometry. In another embodiment, the regions are impenetrable to molecules with a radius of gyration or effective diameter of at least 1,000 Da in size.

In one embodiment, controlled pore collapse is conducted along an axis of the scaffold. In one embodiment, water evaporation from regions of interest may be accomplished at appropriate pressure known in the art, such as, for example, through the use of hot air directed at the region. According to this aspect of the invention, the dried regions will be devoid of pores, or in another embodiment, will be diminished in terms of the extent of porosity in the region, by the controlled collapse of these pores, due to surface tension issues

Such controlled pore closure may be used for creating scaffolding, in another embodiment, for applications where biological baffles are useful In one embodiment, the term “biological baffles” refers to matter, which physically isolates a biological activity in one region from that in an area adjacent thereto.

In one embodiment, such controlled pore closure scaffolds are useful in scaffolding seeded with cells, conferring a particular biological activity, such as described in U.S. Pat. No. 4,458,678 or U.S. Pat. No. 4,505,266. Biological baffles created by controlled pore closure, in one embodiment, creates regions devoid of cells, or, in another embodiment, impenetrable to cells, or in another embodiment, both. Such baffles, in some embodiments, may be useful in separating particular cell types, seeded in the scaffold, or in another embodiment, creating discrete milieu, in separated regions, each with a particular biochemical makeup, such as, for example, regions which vary in terms of the types and/or concentration of cytokines, growth factors, chemokines, etc.

In another embodiment, the process further comprises the step of exposing the scaffold to a gradient of solutions, which are increased in their salt concentration. In one embodiment, exposure to the salt results in selective solubilization of at least one extracellular matrix component in said scaffold. In another embodiment, solubilization of at least one extracellular matrix component increases as a function of increasing salt concentration.

According to this aspect of the invention, and in one embodiment, the gradient scaffold produced may be further influenced by controlling the chemical composition of the resulting scaffold In one embodiment, chemical composition may be controlled by a variation of methods described in U.S. Pat. No. 4,280,954.

For example, and in one embodiment, the scaffold is comprised of a graft copolymer of a type I collagen and a GAG, whose ratio is controlled by adjusting the mass of the macromolecules mixed to form the copolymer.

The complex, in one embodiment, is freeze-dried and sublimated, producing a porous material with a uniform composition, throughout the volume of the solid. In one embodiment, the solid is then exposed to an increasing salt gradient, such as, NaH₂PO₄, or, in another embodiment, NaCl, or in another embodiment, an electrolyte, or in another embodiment, combinations thereof (see fox example, Yannas et al., JBMR, 14:107-0131, 1980).

In one embodiment, the salt solution is at a range corresponding to an ionic strength of between 0.001 and 10. In another embodiment, the salt solution is at a range corresponding to an ionic strength of between 0.001 and 1, or in another embodiment, the salt solution is at a range corresponding to an ionic strength of between 0.01 and 10, or in another embodiment, the salt solution is at a range corresponding to an ionic strength of between 0.1 and 10, or in another embodiment, the salt solution is at a range corresponding to an ionic strength of between 1 and 10, or in another embodiment, the salt solution is at a range corresponding to an ionic strength of between 1 and 20, or in another embodiment, any range in concentration wherein selective solubilization is accomplished, while scaffold integrity is maintained.

In one embodiment, the scaffold is then exposed to water. In another embodiment, solubilization of extracellular matrix components increases as a function of increasing solvent concentration.

The sulfate, in one embodiment, solubilizes the GAG in the solid. In another embodiment, increasing the salt concentration solubilizes GAGs of increased mass, resulting in a gradient in the collagen/GAG ratio.

In another embodiment, the process further comprises the step of exposing the scaffold to a gradient of solutions, which are increased in their concentration of an enzyme, which degrades or solubilizes at least one extracellular matrix component. According to this aspect of the invention, and in one embodiment, digestion of at least one extracellular matrix component increases as a function of increasing enzyme concentration.

In one embodiment, the term degrade/s or solubilizes encompasses partial degradation or solubilization, or in another embodiment, complete degradation or solubilization.

In one embodiment, the enzyme is a collagenase, a glycosidase, or a combination thereof. In one embodiment, the enzyme is an endoglycosidase, which catalyzes the cleavage of a glycosidic linkage. In one embodiment, the endoglycosidase is a Heparitinase, such as, for, example Heparitinase I, II or III. In another embodiment, the endoglycosidase is a Glycuronidase, such as, for example, Δ^{4,5}-Glycuronidase In another embodiment, the glycosidase is an endo-xylosidase, endo-galactosidase, N-glycosidase or an endo-glucuronidase.

In one embodiment, the enzymes are purified, or in another embodiment, from recombinant sources.

In one embodiment, the enzyme concentration is at a range between 0.001-500 U/ml. In another embodiment, the enzyme concentration is at a range between 0.001-500 U/ml, or in another embodiment, enzyme concentration is at a range between 0.001-1 U/ml, or in another embodiment, enzyme concentration is at a range between 0.001-10 U/ml, or in another embodiment, enzyme concentration is at a range between 0.01-10 U/ml, or in another embodiment, enzyme concentration is at a range between 0 01-100 U/ml, or in another embodiment, enzyme concentration is at a range between 0.1-10 U/ml, or in another embodiment, enzyme concentration is at a range between 0.1-100 U/ml, or in another embodiment, enzyme concentration is at a range between 1-10 U/ml, or in another embodiment, enzyme concentration is at a range between 1-100 U/ml, or in another embodiment, enzyme concentration is at a range between 10-100 U/ml, or in another embodiment, enzyme concentration is at a range between 10-250 U/ml, or in another embodiment enzyme concentration is at a range between 10-500 U/ml, or in another embodiment, enzyme concentration is at a range between 100-500 U/ml or in another embodiment, enzyme concentration is at a range between 100-250 U/ml or in another embodiment, enzyme concentration is at a range between 50-100 U/ml or in another embodiment, enzyme concentration is at a range between 50-250 U/ml or in another embodiment, enzyme concentration is at a large between 50-500 U/ml.

In one embodiment, enzyme activity may be determined by any means well known to one skilled in the art. In one embodiment, enzyme degradation of a GAG may be determined by mass spectroscopy, proton and carbon ¹³NMR analysis, or in another embodiment, capillary HPLC-ESI-TOF-MS, high performance liquid chromatography (HPLC), conventional chromatography, gel electrophoresis and the like.

According to this aspect of the invention, and in one embodiment, a gradient scaffold may be prepared by producing a scaffold comprised of a polymer, which is a copolymer, with a specific composition, and in a controlled manner, digesting or solubilizing at least one component of the scaffold, along a particular axis, or according to a desired geometry, thereby producing the gradient scaffold.

In one embodiment, a graft copolymer of two different extracellular matrix components is formed, such as for example a type I collagen and GAG. The final ratio of collagen/GAG may be equal, in another embodiment, to any combination between 85/15 to 100/0 w/w by methods well known in the art (Yannas, et al. PNAS 1989, 86:933)). According to this aspect of the invention, and in one embodiment, a length of the polymer is then exposed to a concentration gradient of a collagenase, for a period of time, wherein time, in another embodiment, is varied, which may, in another embodiment, provide for greater digestion of for example collagen, in some sections of the scaffold thus exposed. In one embodiment, digestion is a function of enzyme concentration, or in another embodiment, exposure time to a given concentration, or in another embodiment, a combination thereof.

In another embodiment, the process further comprises the step of exposing the scaffold to a temperature gradient. According to this aspect of the invention, and in one embodiment, the temperature gradient is a range between 25-200° C. In another embodiment, exposing the scaffold to a temperature gradient, results in the creation of a gradient in crosslink density in said scaffold.

In another embodiment, the process further comprises the step of exposing the scaffold to a gradient of solutions, which are increased in their concentration of cross-linking agent

In one embodiment, cross-link density may be affected via any number of means, well known in the art. According to this aspect of the invention, and in one embodiment, exposure to the cross-linking, agent results in the creation of a gradient in crosslink density in the scaffold.

In one embodiment, gradient scaffolds with varied cross-link density may be accomplished via modifying known methods (for example, Yannas et al., 1980 J. Biomed. Mat. Res. 14: 107-131; Dagalakis et al., 1980 J. Biomed. Mat. Res. 15: 511-528; or U.S. Pat. No. 4,522,753), wherein freeze-dried scaffolds are placed inside a vacuum oven, and exposed to a regimen of temperature, and/or vacuum. Such exposure, in one embodiment, introduces crosslinks in a scaffold comprising collagen and GAG in an ionically complexed form, such as when prepared by precipitation for a solution at acidic pH, as described.

In one embodiment, spatial control of the crosslink density may be accomplished by subjecting the uncrosslinked scaffold in a vacuum to a temperature gradient, for example in a vacuum oven. Such ovens with controlled temperature distribution will be known to one skilled in the art, and may include, for example, installation of heating elements in a particular geometry within the oven, such that one side is heated at a different temperature than the other. According to this aspect of the invention, and in one embodiment, cross-link density is a function of increased temperature

In another embodiment, gradient scaffolds with a gradient in crosslink density may be prepared using a cross linking agent.

In one embodiment, the cross-linking agent is glutaraldehyde, formaldehyde, paraformaldehyde, formalin, (1 ethyl 3-(3dimethyl aminopropyl)carbodiimide (EDAC), or UV light, or a combination thereof. In one embodiment, the concentrations of the crosslinking agents may be the following ranges: glutaraldehyde or formaldehyde, at a range of 0.01-10%; (1 ethyl 3-(3dimethyl aminopropyl)carbodiimide (EDAC) at a range of 0.01-1000 mM; and UV light, at a range of 100-50,000 μW/cm².

In one embodiment, the process may comprise preparing a freeze-dried solid scaffold, and exposing the scaffold to a series of baths with an increasing concentration of the crosslinking agent, such as, for example, glutaraldehyde, or (1 ethyl 3-(3dimethyl aminopropyl)carbodiimide (EDAC), as described. In another embodiment, the freeze-dried scaffold may be exposed to a pressure gradient, such as formaldehyde gas, for example, as describe din U.S. Pat. No. 4,448,718.

In another embodiment, this invention provides a process for preparing a non-uniformly porous, solid, biocompatible scaffold, comprising at least one extracellular matrix component or an analog thereof, comprising the steps of:
(a) Freeze-drying a solution of at least one extracellular matrix component or analogs thereof;
(b) Sublimating ice-crystals formed within the slurry in step (a) to produce a scaffold with uniformly distributed pores;
(c) Moistening at least one region within said scaffold formed in step (b); and
(d) Exposing the moistened region produced in step (c) to drying, under conditions of atmospheric pressure
   Wherein exposing said moistened region to drying results in pole collapse in said region, thereby producing a non-uniformly porous, solid, biocompatible scaffold.

According to this aspect of the invention, and in one embodiment, the process further comprises the step of exposing the scaffold to a gradient of solutions, which ale increased in their salt concentration. In another embodiment, the process further comprises the step of exposing the scaffold to a gradient of solutions, which are increased in their concentration of an enzyme, which degrades or solubilizes at least one extracellular matrix component. In another embodiment, the process further comprises the step of exposing the scaffold to a temperature gradient. In another embodiment, the process further comprises the step of exposing the scaffold to a gradient of solutions, which are increased in their concentration of cross-linking agent.

In another embodiment, this invention provides a process for preparing a solid, porous biocompatible gradient scaffold, comprising at least one extracellular matrix component or an analog thereof, comprising the steps of:
(a) Preparing a solution of a graft copolymer of at least one extracellular matrix component or analogs thereof;
(b) Freeze-drying the solution in step (a) to yield a solid, porous scaffold of uniform composition, and
(c) Exposing the scaffold formed in step (b) to a gradient of solutions, which are increased in their salt concentration;
   Wherein exposing said scaffold to said gradient of solutions, which are increased in their salt concentration results in selective solubilization of at least one extracellular matrix component, and said solubilization increases as a function of increased sulfate salt concentration, thereby producing a solid, biocompatible gradient scaffold.

According to this aspect of the invention, and in one embodiment, the process further comprises the step of exposing the scaffold to a gradient of solutions, which are increased in their concentration of an enzyme, which degrades or solubilizes at least one extracellular matrix component. In another embodiment, the process further comprises the step of exposing the scaffold to a temperature gradient. In another embodiment, the process further comprises the step of exposing the scaffold to a gradient of solutions, which are increased in their concentration of cross-linking agent.

In another embodiment, this invention provides a process for preparing a solid, biocompatible gradient scaffold, comprising one or more extracellular matrix components or analogs thereof comprising the steps of:
(a) Preparing a solution of a graft copolymer of one or more extracellular matrix components or analogs thereof,
(b) Freeze-drying the solution in step (a) to yield a solid, porous scaffold of uniform composition; and
(c) Exposing the scaffold formed in step (b) to a gradient of solutions, which are increased in their concentration of an enzyme which digests at least one of said two or more extracellular matrix components
   Wherein exposing said scaffold to said gradient of solutions, results in selective digestion of at least one of said two or more extracellular matrix components, and said digestion increases as a function of increasing enzyme concentration, thereby producing a solid, biocompatible gradient scaffold

According to this aspect of the invention, and in one embodiment, the process further comprises the step of exposing the scaffold to a temperature gradient. In another embodiment, the process further comprises the step of exposing the scaffold to a gradient of solutions, which are increased in their concentration of cross-linking agent.

In another embodiment, this invention provides a process for preparing a solid, porous biocompatible gradient scaffold, comprising one or more extracellular matrix components or analogs thereof, comprising the steps of:
(a) Preparing a solution of a graft copolymer of two or more extracellular matrix components or analogs thereof; one
(b) Freeze-drying the solution in step (a) to yield a solid porous scaffold of uniform composition; and
(c) Exposing the scaffold formed in step (b) to a temperature gradient
   Wherein exposing said scaffold to said temperature gradient, results in the creation of a gradient in crosslink density in said scaffold, thereby producing a solid, porous biocompatible gradient scaffold.

According to this aspect of the invention, and in one embodiment, the process further comprises exposing the scaffold to a gradient of solutions, which are increased in their concentration of cross-linking agent.

In another embodiment, this invention provides a process for preparing a solid, porous biocompatible gradient scaffold, comprising at least one extracellular matrix component or analogs thereof, comprising the steps of:
(a) Preparing a solution of a graft copolymer of at least one extracellular matrix component or analogs thereof;
(b) Freeze-drying the solution in step (a) to yield a porous, solid scaffold of uniform composition; and
(c) Exposing the scaffold formed in step (b) to a gradient of solutions, which are increased in their concentration of cross-linking agent
   Wherein exposing said scaffold to said gradient of solutions, which are increased in their concentration of cross-linking agent, results in the creation of a gradient in crosslink density in said scaffold, thereby producing a solid, porous biocompatible gradient scaffold.

In another embodiment, this invention provides a gradient scaffold, prepared according to a process of this invention.

It is to be understood that any process of producing a gradient scaffold, or any scaffold produced by a process of this invention, is to be considered as part of this invention.

In one embodiment, small variations in the processes and configurations described herein, enable the formation of scaffolds that are characterized by heterogeneity that varies discontinuously along an axis, in one embodiment, linearly, or in another embodiment, cyclically, or in another embodiment, spatially, according to a specific geometric pattern along one or more axes of the scaffold.

In one embodiment, the gradient may be along two or three axes throughout the scaffold. In one embodiment, such an arrangement may be obtained via control of any or of a number of the parameters listed herein. In one embodiment, the gradient may vary linearly for a given region along one axis, and non-linearly vary, for example, exponentially, along the same axis, at a point distal to the linear region. It is to be understood that all of these represent embodiments of the present invention.

In another embodiment, this invention provides a method of organ or tissue engineering in a subject, comprising the step of implanting a scaffold of this invention in a subject.

In another embodiment, this invention provides a method of organ or tissue repair or regeneration in a subject, comprising the step of implanting a scaffold of this invention in a subject.

According to these aspects of the invention, and in one embodiment, the scaffold may be one produced by a process of this invention.

In one embodiment, the methods of this invention are useful in engineering, repairing or regenerating a connector tissue. The term “connector tissue” refers, in one embodiment to a tissue physically attached to two different tissues, providing a physical connection between them. In one embodiment, the connector tissue fulfills a non-specific connection, such as, for example, the presence of fascia. In another embodiment, the connector tissue confers functional properties, such as for example, tendons, ligament, articular cartilage, and others, where, in one embodiment, proper functioning of one or both tissues thereby connected is dependent upon the integrity, functionality, or combination thereof of the connector tissue.

For example, and in one embodiment, tendon attachment to bone, involves the insertion of collagen fibers (Sharpey's fibers) into the bone The fibers have a distinct architecture, as compared to that of the collagen in the tendon, and in the bone. The mineral structure differs as well, in that tendons are free of hydroxyapatite, however, at regions, which are in closer proximity to the bone, the collagen fibers are calcified, by an increased hydroxyapatite crystal incorporation, and at regions of apposition to bone becomes essentially indistinguishable, in terms of its composition.

In one embodiment, use of the scaffolds for repair, regeneration of tissue is in cases where native tissue is damaged, in one embodiment, by trauma. In one embodiment, the gradient scaffolds of this invention are useful in repairing, regenerating or engineering the connector tissue, and in another embodiment, in facilitating the establishment of physical connections to the tissues, which connector tissue connects. For example, tendon repair, as well as its reattachment to bone may be facilitated via the use of the gradient scaffolds of this invention, and represents an embodiment thereof. In another embodiment, the gradient scaffold allows for incorporation of individual cells, which are desired to be present in the developing/repairing/regenerating tissue,

According to these aspects of the invention, and in one embodiment, the method further comprises the step of implanting cells in the subject. In one embodiment, the cells ale seeded on said scaffold. In another embodiment, the cells are stem or progenitor cells. In another embodiment, the method further comprises the step of administering cytokines, growth factors, hormones or a combination thereof to the subject In another embodiment, the engineered organ or tissue is comprised of heterogeneous cell types. In another embodiment, the engineered organ or tissue is a connector organ or tissue, which in another embodiment, is a tendon or ligament.

As can be seen from the forgoing description, the concepts of the present disclosure provide numerous advantages. For example, the concepts of the present disclosure provide for the fabrication of an implantable gradient scaffold, which may have varying mechanical properties to fit the needs of a given scaffold design. For instance, the pore size and the material density may be varied to produce a scaffold having a desired mechanical configuration. In particular, such variation of the pore size and the material density of the scaffold is particularly useful when designing a scaffold which provides fox a desired amount of cellular migration therethrough, while also providing a desired amount of structural rigidity. In addition, according to the concepts of the present disclosure, implantable devices can be produced that not only have the appropriate physical microstructure to enable desired cellular activity upon implantation, but also has the biochemistry (collagens, growth factors, glycosaminoglycans, etc.) naturally found in tissues where the scaffolding is implanted for applications such as, for example, tissue repair or regeneration.

The following examples serve as a means of instruction for practicing some of the embodiments of the present invention, and are not to be construed as limiting the applications of the present invention in any way.

### EXAMPLES

### Example 1

### Freeze-Sublimation Methods for Constructing Gradient Scaffolding with Varied Pore Diameter

Preparation of Slurry:

Extracellular matrix components, such as, for example, microfibriallar, type I collagen, isolated from bovine tendon (Integra LifeSciences) and chondroitin 6-sulfate, isolated from shark cartilage (Sigma-Aldrich) are combined with 0.05M acetic acid at a pH ˜3.2 are mixed at 15,000 rpm, at 4° C., then degassed under vacuum at 50 mTorr.

Varying Pore Diameter

The suspension is placed in a container, and only part of the container (up to 10% of the length) is submerged in a supercooled silicone bath (Loree et al., 1989) The equilibration time for freezing of the slurry is determined, and the freezing process is stopped prior to achieving thermal equilibrium. The container is then removed from the bath and the slurry is then sublimated via freeze-drying (for example, VirTis Genesis freeze-dryer, Gardiner, N.Y.). Thus, a thermal gradient occurs in the slurry, creating a freezing front, which is stopped prior to thermal equilibrium, at which point freeze-drying is conducted, causing sublimation, resulting in a matrix copolymer with a graded average pore diameter field.

In another method, the suspension is placed in a container, on a freezer shelf, where a graded thermal insulation layer is placed between the container and the shelf, which also results in the production of a gradient freezing front, as described above. The graded thermal insulation layer can be constructed by any number of means, including use of materials with varying thermal conductivity, such as aluminum and copper; or aluminum and plexiglass, and others

### Example 2

### Controlled Pore Closure Methods for Constructing Gradient Scaffolding with Varied Pore Diameter

Preparation of Scaffolding:

Scaffolding is prepared, as in Example 1, with the exception that the slurry is completely immersed in the bath, prior to freeze-drying and sublimation, such that the scaffold comprises a relatively uniform average pore diameter.

Varying Pore Diameter

A region of the prepared scaffolding is moistened, and water is evaporated from this region at the appropriate pressure, for example, via the use of a hot air dryer. Because microscopic pores are subject to high surface tension during the evaporation of water, this leads to pore collapse. The specific pore collapse is controlled, via controlling regions of the scaffolding subjected to pore collapse.

### Example 3

### Solubilization Methods for Constructing Gradient Scaffolding with Varied Chemical Composition

Preparation of Scaffolding:

Scaffolding is prepared from a graft copolymer of type I collagen and a glycosaminoglycan (GAG). Type I collagen and chondroitin 6-sulfate are combined in 0.05M acetic acid at a pH ˜3.2, mixed at 15,000 rpm, at 4° C., and then degassed under vacuum at 50 mtorr. The ratio of collagen/GAG is controlled by adjusting their respective masses used to form the suspension, as described (Yannas et al., 1980 J. Biomedical Materials Research 14: 107-131). The suspension is then freeze-dried and sublimated to create a porous scaffold, with a relatively uniform collagen/GAG ratio throughout the scaffolding.

Varying Chemical Composition

The scaffolding is exposed to an increasing concentration gradient of a salt solution, such as NaH₂SO₄, or NaCl, or electrolytes, which solubilizes the CGAGs, with larger mass GAGs being more readily solubilized, such that a gradient in the collagen/GAG ratio is created along a particular axis. The solution will have an ionic strength of between 0.001 and 10. For further details and examples see Yannas et al., J Biomed Mater Res 14:107-131, 1980]

### Example 4

### Enzymatic Digestion Methods for Constructing Gradient Scaffolding with Varied Chemical Composition

Preparation of Scaffolding:

Scaffolding is prepared from a graft copolymer of type I collagen and a GAG to a final ratio of collagen/GAG of 98/2 w/w, as described (Yannas et al., 1989. Proc. Natl. Acad. Sci. USA, 86, 933-937).

Varying Chemical Composition

Parts of the scaffold are immersed in a series of baths containing an increasing concentration of collagenase (prepared as described in Huang and Yannas, 1977 J. Biomedical Material Research 8: 137-154), which results in increased collagen dissolution from the exposed regions of the scaffolding. Glycosidases may also be used to degrade the GAG component of the scaffold Concentrations of the enzymes used may range from 0.001-500 U/ml.

### Example 5

### Methods for Constructing Gradient Scaffolding with Varied Crosslink Density

Scaffold fabricated from a suspension of collagen and a GAG precipitated from solution with an acidic pH is prepared as has been previously described (Yannas, I. V. et al., 1980 J. Biomedical Material Research 14: 511-528; Yannas et al., PNAS 86(3): 933-937, 1989). The scaffolding is placed in a vacuum oven, and temperate and vacuum conditions in the oven are varied with time, conditions which introduce a varying degree of cross-linking in the scaffolding

Crosslink density in the scaffolding increases with increasing temperature. Temperature can be varied via a number of means, including utilization of an oven with controlled temperature distribution. In some instances the oven may be so constructed to place an electrical heating element in a configuration such that one side is heated to a higher temperature than the other side of the oven, and thus in between a temperature gradient is created The size of the gradient of the crosslink density in the scaffolding can thus be controlled by controlling the temperature gradient in the oven which may range from 25-200° C.

Chemical cross-linking agents may be added to the scaffoldi in a manner to create a gradient cross-link density in the scaffold. One means is via exposing a freeze-dried scaffold as previously described to a series of baths with increasing concentration of a solution of a cross-linking agent such as glutaraldehyde or formaldehyde, at concentrations, in a range such as 0.01-10% or EDAC, at a concentration such as ranging between 0.01-1000 mM EDAC. Another means is via exposing the scaffolding to a gradient of pressurized gas cross-linking agent, such as formaldehyde (see U.S. Pat. No. 4,448,718) or UV light, for example, in a range between 100-50,000 μW/cm².

It will be appreciated by a person skilled in the art that the present invention is not limited by what has been particularly shown and described hereinabove, which serves only as exemplification of some of the embodiments of the present invention.

1. A solid, non-uniformly porous biocompatible gradient scaffold, comprising at least one synthetic or natural polymer, ceramic, metal, extracellular matrix protein or an analogue thereof.

2. (canceled)

3. The gradient scaffold of claim 1, wherein said extracellular matrix proteins comprise a collagen, a glycosaminoglycan, or a combination thereof.

4. The gradient scaffold of claim 3, wherein said glycosaminoglycan is a chondoitin sulfate.

5. (canceled)

6. The gradient scaffold of claim 1, wherein pores within said scaffold are of a non-uniform average diameter.

7. The gradient scaffold of claim 6, wherein the average diameter of said pores ranges from 0.001-500 μm

8. The gradient scaffold of claim 6, wherein said average diameter of said pores varies as a function of its spatial organization in said scaffold.

9. The gradient scaffold of claim 8, wherein said average diameter of said pores varies along an arbitrary axis of said scaffold.

10. The gradient scaffold of claim 1, wherein said scaffold comprises regions devoid of pores.

11. The gradient scaffold of claim 10, wherein said regions are impenetrable to molecules with a radius of gyration or effective diameter of at least 1000 Da in size.

12. The gradient scaffold of claim 6, wherein said scaffold varies in its average pore diameter, or pore size distribution, concentration of components, cross-link density, or a combination thereof.

13. The gradient scaffold of claim 1, wherein said scaffold is characterized by a progressively changing pore volume fraction, ranging from a pore fraction of 0 to 0.999.

14. The gradient scaffold of claim 1, wherein said scaffold varies along a desired direction in the concentration of its components, cross-link density, or a combination thereof.

15. The gradient scaffold of claim 1, wherein the concentration of said polymer in said scaffold varies as a function of its spatial organization in said scaffold.

16. The gradient scaffold of claim 15, wherein said concentration varies along a given direction in said scaffold.

17. The gradient scaffold of claim 1, wherein the crosslink density of said scaffold varies along a desired direction in said scaffold.

18. The gradient scaffold of claim 1, wherein said scaffold further comprises cells, growth factors, cytokines, hormones, or a combination thereof.

19. A process for preparing a non-uniformly porous, solid, biocompatible gradient scaffold, comprising at least one extracellular matrix component or an analog thereof, comprising the steps of:
(a) Freeze-drying a solution of at least one extracellular matrix component or an analog thereof, under conditions producing a gradient in the freezing temperature; and
(b) Sublimating ice-crystals formed within the slurry in step (a), prior to achievement of thermal equilibrium during said freeze-drying;
Wherein ice-crystals are formed along a gradient as a function of the gradient freezing temperature, whereby sublimation of said ice-crystals results in the formation of pores arranged along said gradient.

20. The process of claim 19, wherein said extracellular matrix component comprises a collagen, a glycosaminoglycan, or a combination thereof.

21. The process of claim 20, wherein said glycosaminoglycan is a chondroitin sulfate.

22. (canceled)

23. The process of claim 19, wherein the average diameter of said pores formed ranges from 0.001-500 μm.

24. The process of claim 19, wherein said average diameter of said pores varies as a function of its spatial organization in said scaffold.

25. The process of claim 19, wherein said average diameter of said pores varies along an arbitrary axis of said scaffold.

26. The process of claim 19, further comprising the steps of moistening at least one region within said scaffold formed in step (b) and exposing the moistened region to drying, under appropriate conditions for conversion of liquid water to water vapor, such that exposing said moistened region to drying results in pore collapse in said region.

27. The process of claim 26, wherein said scaffold produced comprises regions devoid of pores.

28. The process of claim 26, wherein moistening said region is conducted such that following exposure to said drying, said regions devoid of pores assume a particular geometry or pattern.

29. (canceled)

30. The process of claim 19, further comprising the step of exposing the scaffold to a gradient of solutions, wherein said solutions are characterized by increasingly higher salt concentration.

31. The process of claim 30, wherein exposure to said salt results in selective solubilization of at least one extracellular matrix component in said scaffold.

32. The process of claim 30, wherein solubilization of said at least one extracellular matrix component increases as a function of increasing salt concentration.

33. The process of claim 30, wherein said salt concentration is in a range corresponding to an ionic strength of between 0.001 and 10.

34. The process of claim 30, wherein said salt is Na₂PO₄, NaCl or combinations thereof

35. The process of claim 30, wherein the scaffold is exposed to water.

36. The process of claim 35, wherein solubilization of said at least one extracellular matrix component increases as a function of increasing solvent concentration

37. The process of claim 19, further comprising the step of exposing the scaffold to a gradient of solutions, comprising solutions of increasing concentration of an enzyme, which degrades or solubilizes at least one extracellular matrix component

38. The process of claim 37, wherein solubilization or degradation of said at least one extracellular matrix component increases as a function of increasing enzyme concentration.

39. The process of claim 37, wherein said enzyme is a coil agenase, a glycosidase, or a combination thereof.

40. The process of claim 37, wherein said enzyme concentration is in a range between 0.001-500 U/ml

41. The process of claim 19, further comprising the step of exposing the scaffold to a temperature gradient

42. The process of claim 41, wherein said temperature gradient is a range between 25-200° C.

43. The process of claim 41, wherein exposing said scaffold to said temperature gradient, results in the creation of a gradient in crosslink density in said scaffold.

44. The process of claim 19, further comprising the step of exposing the scaffold to a gradient of solutions, which are increased in their concentration of cross-linking agent.

45. The process of claim 44, wherein exposure to said cross-linking agent results in the creation of a gradient in crosslink density in said scaffold.

46. The process of claim 44, wherein said cross-linking agent is glutaraldehyde, formaldehyde, paraformaldehyde, formalin, (1 ethyl 3-(3dimethyl aminopropyl)carbodiimide (EDAC), or UV light, or a combination thereof.

47. A non-uniformly porous, solid, biocompatible gradient scaffold prepared according to the process of claim 19.

48-100. (canceled)

101. A process for preparing a solid, porous biocompatible gradient scaffold, comprising at least one extracellular matrix components or analogs thereof, comprising the steps of:
(a) Preparing a solution of a graft copolymer of at least one extracellular matrix components or analogs thereof;
(b) Freeze-drying the solution in step (a) to yield a solid, porous scaffold of uniform composition; and
(c) Exposing the scaffold formed in step (b) to a temperature gradient
Wherein exposing said scaffold to said temperature gradient, results in the creation of a gradient in crosslink density in said scaffold, thereby producing a solid, porous biocompatible gradient scaffold.

102. The process of claim 101, wherein said extracellular matrix components comprise a collagen, a glycosaminoglycan, or a combination thereof.

103. The process of claim 101, wherein said glycosaminoglycan is a chondroitin sulfate.

104. The process of claim 101, wherein said temperature gradient is a range between 25-200° C.

105. The process of claim 101, further comprising the step of exposing the scaffold to a gradient of solutions, which are increased in their concentration of cross-linking agent.

106. The process of claim 105, wherein exposure to said cross-linking agent results in the creation of a gradient in crosslink density in said scaffold.

107. The process of claim 105, wherein said cross-linking age is glutaraldehyde, (1 ethyl 3-(3dimethyl aminopropyl)carbodiimide (EDAC), formaldehyde, paraformaldehyde, UV light of intensity sufficient to induce crosslinking or a combination thereof.

108. A solid, biocompatible gradient scaffold, prepared according to the process of claim 101.

109. A process for preparing a solid, porous biocompatible gradient scaffold, comprising at least one extracellular matrix component or analogs thereof, comprising the steps of:
(d) Preparing a solution of a graft copolymer of one or more extracellular matrix components or analogs thereof;
(e) Freeze-drying the solution in step (a) to yield a solid, in porous scaffold of uniform composition; and
(f) Exposing the scaffold formed in step (b) to a gradient of solutions, which are increased in their concentration of cross-linking agent
Wherein exposing said scaffold to said gradient of solutions, which are increased in their concentration of cross-linking agent, results in the creation of a gradient in crosslink density said scaffold, thereby producing a solid, porous, biocompatible gradient scaffold.

110. The process of claim 109, wherein extracellular matrix components comprise a collagen, a glycosaminoglycan, or a combination thereof.

111. The process of claim 110, wherein said glycosaminoglycan is a chondroitin sulfate.

112. The process of claim 109, wherein said cross-linking agent is glutaraldehyde, (1 ethyl 3-(3dimethyl aminopropyl)carbodiimide (EDAC), formaldehyde, paraformaldehyde, UV light or a combination thereof.

113. A solid, biocompatible gradient scaffold, prepared according to the process of claim 109.

114. A method of organ or tissue engineering in a subject, comprising the step of implanting a scaffold of claim 1 in said subject.

115. The method of claim 114, further comprising the step of implanting cells in said subject.

116. The method of claim 115, wherein said cells are seeded on said scaffold.

117. The method of claim 115, wherein said cells are stein or progenitor cells.

118. The method of claim 114, further comprising the step of administering cytokines, growth factors, hormones or a combination thereof.

119. The method of claim 114, wherein the engineered organ or tissue is comprised of heterogeneous cell types.

120. The method of claim 114, wherein the engineered organ or tissue is a connector organ or tissue.

121. The method of claim 120, wherein said connector tissue is a tendon or ligament.

122. A method of organ or tissue repair or regeneration in a subject, comprising the step of implanting a scaffold of claim 1 in said subject.

123.-129. (canceled)
